(19) United States
(12) Patent Application Publication
(10) Pub. No.: US 2020/0399650 A1
(43) Pub. Date: Dec. 24, 2020

(54) METHODS FOR GENERATING CROP YIELD TRAITS

(71) Applicant: EnobraQ, Ramonville St-Agne (FR)

(72) Inventors: Cedric BOISART, Belberaud (FR); Nicolas CHABOT, Ramonville St-Agne (FR)

(21) Appl. No.: 16/907,033

(22) Filed: Jun. 19, 2020

Related U.S. Application Data
(60) Provisional application No. 62/864,759, filed on Jun. 21, 2019.

(51) Int. Cl. C12N 15/82; C12N 15/10

(52) U.S. Cl. CPC C12N 15/8261; C12N 15/1082

(57) ABSTRACT

The present technology relates to a method for generating crop yield traits. The method comprises identifying a multi-genic system associated with one or more gene of interest, wherein the one or more gene of interest is operatively linked to yield of crop. Identifying at least one combination of genes within the multi-genic system that yields an active multi-protein system of interest and generating mutational diversity on the at least one combination of genes to generate a library. The method further comprises screening the library to identify mutations yielding an improved activity of multi-protein system of interest in an expression system and introducing the screened mutations into the crop.

### CROSS-REFERENCE TO RELATED APPLICATION

This application claims the benefit of and priority to U.S. provisional patent application No. 62/864,759, filed on Jun. 21, 2019; the content of all of which is herein incorporated in entirety by reference.

### FIELD OF TECHNOLOGY

The present technology generally relates to methods for generating crop yield traits.

### BACKGROUND INFORMATION

Global agricultural demand is rapidly increasing as the world's population reaches 9 billion humans by the middle of the century (FAOSTAT 2016). According to projections, agricultural production will have to increase by 70 to 100% to meet this demand (Ray, Mueller, West, & Foley, 2013; Tilman, Balzer, Hill, & Befort, 2011), even while available arable land is stagnating or even decreasing. Selective cultivation and increased inputs (fertilizers) have allowed global agricultural production to double, while improving yield potential and resistance to environmental and biotic stresses. It is necessary to improve crop plants and cropping systems to meet the challenge of doubling production, improving yield potential must play a central role.

To date, yield traits generation in crop by methods as transgenesis and breeding, are very long and limited. These methods only target a single gene at a time.

Crop yield depends directly on several environmental factors, including, but not limited to, CO₂ capture, water use efficiency, light energy, nitrogen fixation and temperature. Each of these factors is governed by dedicated subsystems and mostly multi-genic systems. By example, the enzyme that binds carbon dioxide from the Calvin cycle in photosynthetic organisms, the Ribulose-1,5-bisphosphate carboxylase/oxygenase (RuBisCO, E.C. 4.1.1.39). RuBisCO is a multi-genic system composed by large (L) and small (S) subunits of Rubisco, along with a network of seven compatible auxiliary proteins.

Due to its key position in biomass production, RuBisCO is important for agriculture. For several reasons, it is widely accepted that improving RuBisCO activity will lead to a significant increase in crop productivity. First, the reaction catalysed by RuBisCO limits the growth rate of plants under optimal growth conditions (high temperature and light intensity, abundant nitrogen). Second, compared with many other enzymes, RuBisCO appears to be an ineffective catalyst that leaves much room for optimization (Whitney, Houtz, & Alonso, 2011). A longstanding target for improving crop productivity is to enhance the CO₂-fixing properties of the RuBisCO. Despite 50 years of effort, success has been limited. Food production requires application of fertilizers containing phosphorus, nitrogen and potassium on agricultural fields in order to sustain crop yields. Nitrogen (N) availability is a major parameter determining productivity in plants. Accordingly, genetically improved N₂ fixation may increase plants productivity. Biological nitrogen (N) fixation, a process unique to diazotrophic bacteria, is catalyzed by the nitrogenase complex. Catalytic nitrogenase (Nif) proteins from the diazotroph Klebsiella pneumoniae or from Azotobacter vinelandii can be individually expressed in S. cerevisiae to carry out mutagenesis improving nitrogenase efficiency. Nitrogenase activity improvement affected seed yield more than seed protein content. A functional nitrogenase needs to express simultaneously Nif proteins B, D, E, F, H, J, K, M, N, Q, S, U, V, X, Y, and Z. Modern agriculture is dependent on phosphorus derived from phosphate rock, which is a non-renewable resource and current global reserves may be depleted in 50-100 years. Inorganic phosphate concentration in plant tissues has been measured at 5-20 mM (Raghothama, 1999), whereas the level available in soils is typically less than 10 μM (Bieleski, 1973). This sharp concentration gradient between the plant and the soil illustrates the crucial role of Pi transporters. High-affinity phosphate transporter (PHT1) have been identified in Arabidopsis thaliana in 1996 and have been expressed successfully in S. cerevisiae (Muchhal et al., 1996). Previous results (Mitsukawa et al. 1997) indicate that overexpression of phosphate transporters PHT1 from Arabidopsis thaliana in tobacco cultured cells enhances cells growth under phosphate-limited conditions, establishing gene engineering of phosphate transporter multigenic family as one approach toward enhancing plant cell growth.

In view of this, there remains a need in the field for methods for generating crop yield traits.

### SUMMARY OF DISCLOSURE

According to various aspects, the present technology provides for methods allowing working multi-genic system in yeast and generating yield traits of system.

According to various aspects, the present technology relates to a method for generating yield traits to improve crop yield, the method comprising: identifying a multi-genic system associated with one or more gene of interest, wherein the one or more gene of interest is operatively linked to crop yield; expressing in an expression system a plurality of combinations of genes from crop that are homologous to the multi-genic system to obtain a first library; screening the first library for at least one combination of genes yielding the active multi-genic system of interest in the expression system; integrating the at least one combination of genes in the expression system to carry out an adaptive laboratory evolution and to generate an evolved population of the expression system; screening the evolved population to identify mutations resulting in an improved activity of the multi-genic system of interest; and introducing the screened mutations of into the crop.

According to various aspects, the present technology relates to a method for generating crop yield traits, the method comprising: identifying a plant multi-genic system associated with one or more genes of interest, wherein the one or more genes of interest are operatively linked to crop yield; expressing in yeast a plurality of combinations of genes that are homologous to the plant multi-genic system to obtain a first library; screening the first library for at least one combination of genes yielding the desired activity in yeast; generating mutational diversity on the at least one gene of the combination of genes to generate a second library; screening the second library to identify mutations yielding an improved activity in yeast; and introducing the characterized mutations into the crop.

According to various aspects, the present technology relates to a method for generating crop yield traits, the method comprising: identifying a plant multi-genic system associated with one or more genes of interest, wherein the one or more genes of interest are operatively linked to crop yield; expressing in yeast a plurality of combinations of genes that are homologous to the plant multi-genic system to obtain a first library; screening the first library for at least one combination of genes yielding the desired activity in yeast; generating mutational diversity on the at least one gene of the combination of genes to generate a second library in a first yeast; generating mutational diversity on the at least one different gene of the combination of genes to generate a third library in a second yeast; mixing both libraries by mating of first and second yeast, screening the mixed libraries to identify mutations yielding an improved activity in yeast; and introducing the characterized mutations into the crop.

According to various aspects, the present technology relates to a method for generating crop yield traits to improve crop yield, the method comprising: identifying a plant multi-genic system associated with one or more genes of interest, wherein the one or more genes of interest are operatively linked to crop yield; expressing in yeast a plurality of combinations of genes from crop that are homologous to the multi-genic system to obtain a library; screening the library for at least one combination of genes yielding the desired activity in yeast; integrating at least one combination of genes in yeast to carry out an adaptive laboratory evolution and to generate an evolved yeast population; screening the evolved yeast population to identify mutations resulting in an improved activity of the multi-genic system of interest; and introducing the screened mutations into the crop.

Other aspects and features of the present disclosure will become apparent to those ordinarily skilled in the art upon review of the following description of specific embodiments.

### BRIEF DESCRIPTION OF FIGURES

All features of embodiments which are described in this disclosure are not mutually exclusive and can be combined with one another. For example, elements of one embodiment can be utilized in the other embodiments without further mention. A detailed description of specific embodiments is provided herein below with reference to the accompanying drawings in which:

FIG. 1 is a schematic representation of a multi-genic system according to one embodiment of the present technology.

FIG. 2 is a schematic representation of cloning of bloc of expression unit, insertion into yeast chromosome then mating, according to another embodiment of the present technology.

FIG. 3 is a schematic representation of yeast matting with different heterologous gene inserted in a different chromosome.

### DETAILED DISCLOSURE OF EMBODIMENTS

The present technology is explained in greater detail below. This description is not intended to be a detailed catalog of all the different ways in which the technology may be implemented, or all the features that may be added to the instant technology. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which variations and additions do not depart from the present technology. Hence, the following description is intended to illustrate some particular embodiments of the technology, and not to exhaustively specify all permutations, combinations and variations thereof.

As used herein, the singular form “a,” “an” and “the” include plural referents unless the context clearly dictates otherwise.

The recitation herein of numerical ranges by endpoints is intended to include all numbers subsumed within that range (e.g., a recitation of 1 to 5 includes 1, 1.25, 1.5, 1.75, 2, 2.45, 2.75, 3, 3.80, 4, 4.32, and 5).

The term “about” is used herein, explicitly or not; every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. For example, the term “about” in the context of a given value or range refers to a value or range that is within 20%, preferably within 15%, more preferably within 10%, more preferably within 9%, more preferably within 8%, more preferably within 7%, more preferably within 6%, and more preferably within 5% of the given value or range.

The expression “and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein. The term “or” as used herein should in general be construed non-exclusively. For example, an embodiment of “a composition comprising A or B” would typically present an aspect with a composition comprising both A and B. “Or” should, however, be construed to exclude those aspects presented that cannot be combined without contradiction (e.g., a composition pH that is between 9 and 10 or between 7 and 8).

As used herein, the term “comprise” is used in its non-limiting sense to mean that items following the word are included, but items not specifically mentioned are not excluded.

As used herein, the term “crop” relates to a plant product that can be grown and harvested extensively for profit or subsistence. Crop may refer either to the harvested parts or to the harvest in a more refined state. Most crops are cultivated in agriculture or aquaculture. A crop may be expanded to include macroscopic fungus (e.g. mushrooms), or alga (algaculture).

As used herein, the expression “crop yield” or “agricultural output” refers to both the measure of the yield of a crop per unit area of land cultivation, and the seed generation of the plant itself. Examples of crop yield include, but are not limited to: growth, biomass, crop yield, seed yield, increased cell proliferation, increased organ, cell size and increased total plant mass, or the like. “Yield” or “crop yield” refers also to the measurement of photosynthesis, the measurement of the amount of a crop that has been harvested per unit area of land. Crop yield is also the measure often used for cereals and generally corresponds to the amount of plants harvested per unit area over a given period, i.e. in metric tons per hectare or in kilograms per hectare. Crop yield can also refer to the seed or biomass produced or generated by the plant. Crop yield depends directly on several environmental factors, including, but not limited to, photosynthesis efficiency, CO₂ capture, water use efficiency, light energy, nitrogen fixation and temperature.

“Yield trait” or “Yield traits” includes, but is not limited to, increasing yield, notably increasing yield under non-stress conditions and increasing yield under environmental stress conditions. Stress conditions may include, for example, drought, shade, fungal disease, viral disease, bacterial disease, insect infestation, nematode infestation, cold exposure, heat exposure, reduced availability of nitrogen nutrients, phosphorus and high plant densities. Many agronomic traits can influence “yield”, notably plant height, stem strength, root strength, stem diameter, stem volume, wood density, stem dry weight, bark dry weight, average internode length, internode number, vegetative growth, biomass production, seed production, pod number, pod position on the plant, the incidence of pod shatter, seed size, nodulation and nitrogen fixation efficiency, nutrient uptake efficiency, resistance to biotic and abiotic stress, carbon uptake, plant architecture, lodging resistance, seed germination percentage, seedling vigor and juvenile yield traits. The other characteristics that may affect yield are notably germination efficiency (including in harsh conditions), growth rate (including growth rate in harsh conditions), number of ears, number of seeds per ear, seed size, seed composition (starch, oil, protein, moisture content) and seed filling characteristics. The generation of transgenic plants with desirable phenotypic properties, which may or may not confer an increase in overall plant yield, is also of interest. These properties include improved plant morphology, plant physiology or improved components of the mature seed harvested from the genetically modified plant.

“Plant subsystem” or “Plant subsystem of interest” refers to dedicated multi-genic systems that govern several environmental factors, including, but not limited to, photosynthesis efficiency, CO₂ capture, water use efficiency, light energy, nitrogen fixation and temperature. By example, the multi-genic system that express the enzyme that binds carbon dioxide from the Calvin cycle in photosynthetic organisms is the Ribulose-1,5-bisphosphate carboxylase/oxygenase (RuBisCO, E.C. 4.1.1.39). RuBisCO is a multi-genic system composed by large (L) and small (S) subunits of Rubisco, along with a network of seven compatible auxiliary proteins.

In some embodiments, the present technology relates to a plant of interest that is to be ameliorated or improved.

In some embodiments, the present technology relates to a method for improving a plant subsystem. In some implementations of these embodiments, the method comprises performing a bioinformatics analysis to identify in the plant of interest, the genes that are homologous to the genes of interest. The gene homologs that are identified are referred to as forming a multi-genic system for the plant subsystem of interest.

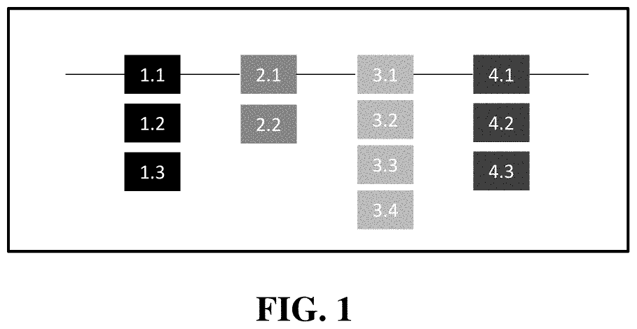

FIG. 1 shows a representation of a multi-genic system for a plant subsystem of interest. An example of a multi-genic system expressing a plant subsystem that could be targeted by the methods of the present technology is the system allowing to a functional RuBisCO expression in E. coli (Aigner et al., 2017). In order to fold correctly subunit L and S of RuBisCO, 7 different chaperonins are required: Cpn60α, Cpn60β, Cpn20, RbcX, Raf1 Raf2 and Bsd2.

In some embodiments, the present technology relates to methods for generating crop yield traits. In some instances, the crops exhibit less than optimal (e.g., sub-optimal) yield, and there is a desire to increase the yield of these crops.

In some embodiments, the method comprises identifying the genes in the plant (“genes of interest”) that are associated with the manifestation of the desired activity.

The methods of the present technology could be applied to any plant species, including, but not limited to, monocots and dicots. Examples of plant species of interest include, but are not limited to Arabidopsis thaliana, tobacco (Nicotiana tabacum), maize (Zea mays), tomato (Solanum lycopersicum), rice (Oryza sativa), wheat (Triticum aestivum), soybean (Glycine max), cucumber (Cucumis sativus), sunflower (Helianthus annuus), rapeseed (Brassica napus L.), canola, Brassica sp. (for example, B. napus, B. rapa, B. juncea), in particular the Brassica species useful as sources of seed oil, alfalfa (Medicago sativa), barrel medic (Medicago truncatula), rye (Secale cereale), sorghum (Sorghum bicolor, Sorghum vulgare), millet (e.g., millet (Pennisetum glaucum), common millet (Panicum miliaceum), foxtail millet (Setaria italica), finger millet (Eleusine coracana), saffron (Carthamus tinctorius), potato (Solanum tuberosum), peanut (Arachis hypogaea), camelina (Camelina sativa), cotton (Gossypium barbadense, Gossypium hirsutum), sweet potato (Ipomoea batatas), cassava (Manihot esculenta), coffee (Coffea spp.), coconut (Cocos nucifera), pineapple (Ananas comosus), citrus fruits (Citrus spp.), cocoa (Theobroma cacao), tea (Camellia sinensis), banana (Musa spp.), avocado (Persea americana), fig (Ficus carica), guava (Psidium guajava), mango (Mangifera indica), olive (Olea europaea), papaya (Carica papaya), cashew nut (Anacardium occidentale), macadamia nut (Macadamia integrifolia), almond (Prunus amygdalus), sugar beet (Beta vulgaris), sugar cane (Saccharum spp.), pigeon pea (Cajanus cajan), chickpea (Cicer arietinum), Cucurbitaceae (Cucurbita sp.) such as squash (Cucurbita pepo L.) and pumpkin (Cucurbita maxima subsp. maxima), brown mustard (Brassica juncea), ryegrass (Lolium sp.), beans (Phaseolus spp.), oil palm (Elaeis guineensis Jacq.), apple (Malus domestica), vine (for example Vitis vinifera), onion (Allium cepa), oat, barley (Hordeum vulgare), legume, glasswort (Salicornia sp.), fruits and vegetables, such as blackberry, blueberry, strawberry and raspberry, cantaloupe, carrot, eggplant, grape, lettuce, mango, melon, papaya, peppers, spinach, woody species such as pine, poplar and eucalyptus, mint, rubber tree, ornamental plants and conifers.

In some embodiments, the method of the present technology further comprises determining the ensemble of plant genes that are homologous to the genes of interest. The ensemble of plant genes homologous to the genes of interest is herein referred to as “multi-genic system”.

In some embodiments, the method further comprises creating a library of plant genes that are homologous variants of the genes of the multi-genic system and expressing such library in yeast. The library may then be screened in yeast for combinations of plant genes that, when expressed, allows for functional expression of the multi-protein system, and selecting such combinations.

In some embodiments, the method further comprises generating diversity on selected genes and screening the library to identify combination of variants resulting in an improved sub-system in yeast. The mutated genes may then be sequenced and characterized and then transferred back into the plant of interest by introducing the same mutations in plant genes by gene editing or transgenesis technologies. The mutated plant phenotype may be characterized in a controlled greenhouse environment and the yield improvement may be evaluated in field trials.

i) Identification of a Multi-Genic System of Interest

In some embodiments, the methods of the present technology comprises identifying a gene of interest or a set of genes of interest that are responsible for the manifestation of a certain activity of a plant subsystem of interest in the crop (“plant subsystem of interest”). In some instances, the plant subsystem of interest is a plant subsystem that is to be ameliorated or improved.

The method comprises performing a bioinformatics analysis to identify in the plant of interest, the genes that are homologous to the genes of interest. The gene homologs that are identified are referred to as forming a multi-genic system for the plant subsystem of interest. FIG. 1 shows a representation of a multi-genic system for a plant subsystem of interest. An example of a multi-genic system expressing a plant subsystem that could be targeted by the methods of the present technology is the system allowing to a functional RuBisCO expression in E. coli (Aigner et al., 2017). In order to fold correctly subunit L and S of RuBisCO, 7 different chaperonins are required: Cpn60α, Cpn60β, Cpn20, RbcX, Raf1 Raf2 and Bsd2.

The homologous genes can be identified by sequence alignment. The procedures for aligning a sequence comparison are well known to the skilled person. In some instances, a mathematical algorithm can be used to determine the percentage of sequence identity between two sequences. Non-limiting examples of algorithms include: the mathematical algorithm of Myers and Miller (Myers & Miller, 1988), the CABIOS algorithm (Grice, Hughey, & Speck, 1997; Wheeler & Hughey, 2000), the global alignment method of Needleman and Wunsch (Needleman & Wunsch, 1970; W R Pearson & Lipman, 1988), the local alignment method (Karlin & Altschul, 1990, 1993), all of which are incorporated by reference herein. The computer versions of these mathematical algorithms can be used for sequence comparison to determine sequence identity. These include, but are not limited to, the Geneious® program purchased from Biomatters (New Zealand), the MultiAlin online program (Corpet, 1988) (http://multalin.toulouse.inra.fr/multalin/), the T-Coffee online program (http://tcoffee.crg.cat/) which is a multiple sequence alignment package. T-Coffee can be used to align sequences or to combine the output of alignment methods (Clustal, Mafft, Probcons, Muscle, or the like) into a single alignment (M-Coffee). The default settings to use are the alignments using programs from the following examples: CLUSTAL program (Corpet, 1988; Higgins & Sharp, 1988, 1989; Huang, Miller, Schwartz, & Hardison, 1992; William R. Pearson, 1994), the program of Myers and Miller (Myers & Miller, 1988) and the BLASTP BLAST protein search program (score=100, word length=12), to obtain protein sequences homologous to a protein sequence of the present invention, in order to obtain split alignments (for comparison purposes), such as those described by Altschul et al., 1997 (see blast.ncbi.nlm.nih.gov).

ii) Reconstruction the Multi-Genic System in Yeast and Selection for Combination Yielding Improved Desired Activity

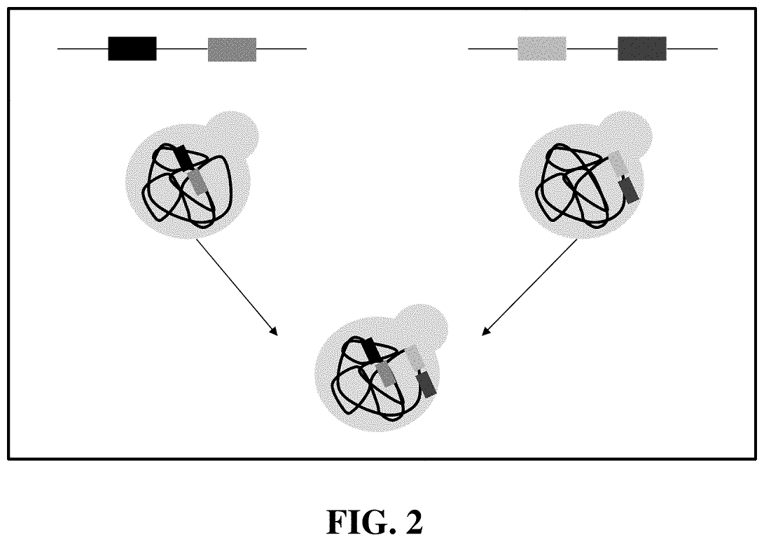

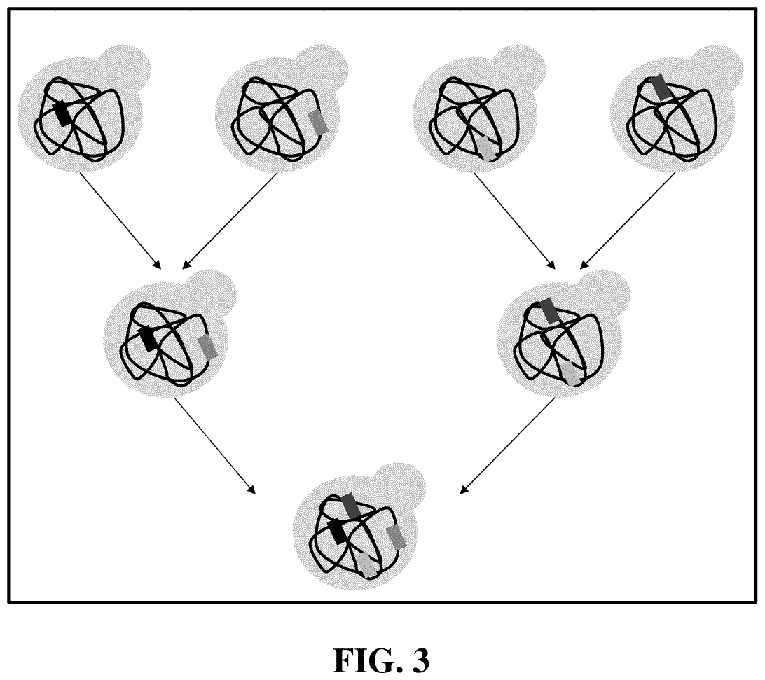

In some embodiments, the method of the present technology comprises expressing various combinations of the genes of the multi-genic system in an expression system (e.g., a yeast system). In some instances, this step involves expressing the homologs of the plant genes of interest in the expression system. This system may be constructed in different ways which are known in the art. For example, each of the genes of the multi-genic system may be cloned in an expression unit (e.g., the expression unit comprising: promoter, gene and terminator). Each expression unit may be assembled in a bloc which may then be inserted into the chromosomes of the expression system. In order to combine each bloc, a mating of different strains is performed (FIG. 2). In another example, each gene of the multi-genic system may be inserted into different chromosomes in the expression system. The total combination of genes will be achieved by mating of the different expression system (FIG. 3).

In a further example wherein the expression system is yeast, each gene of the multi-genic system may be inserted into different yeast chromosome using the CRISPR/Cas9 methodology.

In a further example, each gene of the multi-genic system may be inserted into one or different chromosomes in yeast or each gene of the multi-genic system may be expressed on one or more plasmids. Following the insertion of the genes into the yeast chromosome, the combination of genes is achieved by mating the yeasts such as illustrated in FIG. 3. This combination step allows to create a library of different gene combinations. The library may then be screened to find at least one combination of genes that result in functional plant system in the yeast, and by electing such combination for further testing.

Analysis of the resulting activity may be achieved by in vitro or in vivo enzymatic assays which will be chosen based in part of the nature of the plant subsystem to be assessed. In some embodiments, diversity on selected genes is generated in vivo or in vitro by techniques such as, but not limited to: rational and/or semi-rational and/or random directed evolution (Gonzalez-Perez, Garcia-Ruiz, & Alcalde, 2012; Vifla-Gonzalez, Gonzalez-Perez, & Alcalde, 2016), adaptive laboratory evolution (ALE) (Heidenreich, 2007), random mutagenesis. According to an embodiment, the rational and/or semi-rational and/or random directed evolution is carried out in vivo or in vitro by example, but not limited to: error prone PCR, MORPHING, shuffling, orthogonal replication system (OrthoRep) (Javanpour & Liu, 2019), Yeast Oligo-Mediated Genome Engineering (YOGE) (DiCarlo et al., 2013). According to an embodiment, the gene combination previously determined is integrated into yeast engineered in order to constrain the yeast metabolism to use the protein expressed by the combined genes. Once the yeast thus modified is obtained, a step of adaptive laboratory evolution (ALE) is carried out. During this step, selection pressure leads to an evolution of the strain due to generation of spontaneous mutations. Random mutagenesis is performed during adaptive laboratory evolution directed evolution in order to increase mutation generation by UV treatment and/or mutagenesis agent such as, but not limited to: N-methyl-N′-nitro-N-nitrosoguanidine (NTG), Ethyl Methyl Sulfate (EMS), or the like.

In another embodiment of this method, site-directed mutagenesis is performed on at least one gene or on several genes which form part of the selected combination. A bioinformatics study is performed to identify the amino-acids or amino-acids group to be targeted. For RuBisCOs important amount of biochemical and kinetic data has been derived from various organisms (286 different) (Flamholz et al., 2019) and of different forms. RuBisCOs from plant are hetero-multimeric enzyme composed of RbcL and RbcS subunits (RbcL)8(RbcS)8. Different part of RbcL and/or RbcS will be targeted.

From various databases, the kinetic tradeoffs that can exist between the parameters for the oxygenase and carboxylase activities have been studied. For example for RuBisCO enzyme, that amount of data is of significant interest to study the relationships between the primary RbcL/S structures and the kinetic parameters. For oxygenase and carboxylase reactions, several key enzymatic parameters are often reported. It is i) the turn-over number (k_{cat,C} and k_{cat,O} for carboxylase and oxygenase activities, respectively), ii) the affinity for its sugar-phosphate substrate (RuBP) (K_{RuBP}), iii) the affinities for the gaseous molecules (Michaelis constants, K_{C} and K_{O} for CO₂ and O₂, respectively) and the S_{C/O} value that represents the ratio of catalytic efficiencies toward CO₂ and O₂ (S_{C/O}=(k_{cat,C}/K_{C})/(k_{cat,O}/K_{O})). This last value (S_{C/O}) can also be calculated at O₂ atmospheric conditions (S_{C/O}^{21% O2}).

By combining in-house bioinformatics for primary structure alignments and dimensional reduction, an ANOVA-type analysis is carried out to identify correlations between combinations of mutations with modulations of kinetic parameters in order to improve carboxylase reaction and decrease oxygenase reaction.

In some embodiments, site-directed mutagenesis is generated in vivo or in vitro by techniques such as, but not limited to: site saturation, error prone PCR, and combinatorial library. This method allows crossing different libraries. In order to screen the generated mutant libraries and/or the crossing of these mutant libraries, an original screening was developed. This screening makes it possible to discriminate the most active proteins in vivo. If the protein expressed via the multi-genic system has one or more distinct activity, the screening discriminates each activity in vivo simultaneously, which characterizes the best combination of mutations which result in an improved activity. At the end of the directed evolution or after random mutagenesis or after directed mutagenesis, one or all of the genes of the multi-genic system is/are sequenced and the activity of the protein resulting from the combination of genes is determined.

The evolved yeast is sequenced and the activity of the protein resulting from the combination of genes is determined. Characterization of mutation one by one or in combination is carried out to evaluate if the effect of mutation is individual or synergetic.

Mutations identified as being responsible for the improvement or amelioration of a plant subsystem are introduced into the plant of interest by gene/base editing.

The methods for replacing codons in plant genes are well-known to the skilled person, notably by the use of precise genome editing technologies to modify the endogenous sequence. These methods include, but are not limited to, meganucleases designed against the plant genomic sequence of interest (D'Halluin et al., 2013), CRISPR-Cas9 (Jaganathan, Ramasamy, Sellamuthu, Jayabalan, & Venkataraman, 2018; Jiang et al., 2013), CRISPR-Cpfl (X. Li et al., 2018; Zaidi, Mahfouz, & Mansoor, 2017; Zetsche et al., 2016), TALEN and other technologies for precise genome editing (Patent No. WO/2013/026740, 2013; Feng et al., 2013; Podevin, Davies, Hartung, Nogue, & Casacuberta, 2013; Qi et al., 2016; Zetsche et al., 2015), Argonaute-mediated DNA insertion (Gao, Shen, Jiang, Wu, & Han, 2016), Cre-lox site-specific recombination (Albert, Dale, Lee, & Ow, 1995; Lyznik, Gordon-Kamm, & Tao, 2003), FLP-FRT recombination (Z. Li et al., 2009); Bxbl-mediated integration (Yau, Wang, Thomson, & Ow, 2011), zinc finger induced integration (Cai et al., 2009; Wright et al., 2005) and homologous recombination (Lieberman-Lazarovich & Levy, 2011; Puchta, 2002). Mention may also be made of the patents and patent applications U.S. Pat. No. 9,840,699, US 2013/0321210, EP 3 216 687 and the publication by Nishida et al., 2016.

The mutation may be introduced by transgenesis technologies, for example, by replacing all or part of the coding sequence of the native gene, notably using known homologous recombination techniques. Particular mention may be made of targeted gene modification techniques, notably for introducing point mutations, which are also known to the skilled person, notably described in reference works such as Advances in New Technology for Targeted Modification of Plant Genomes (Zhang et al., 2015) and Site-directed insertion of transgenes (Renault and Duchateau, 2012).

Characterization of the yield traits thus generated may be carried out in a greenhouse to determine the crops yield improvement. Yield is normally defined as the measurable product of the economic value of a crop. This can be defined in terms of amount and/or quality. Yield depends directly on several plant subsystems, such as organelle number and size, plant architecture (for example, number of branches), seed production, leaf senescence, root development, nutrient absorption, stress tolerance, photosynthetic carbon uptake rates and early vigor can also be important yield traits in determining yield. Optimizing and improving the above-mentioned plant subsystems can therefore help to increase crop yield. Yield increase can be characterized by increasing the plant's yield under stress-free conditions or increasing the plant's yield under one or more environmental stress conditions, including, but not limited to, water stress, cold stress, heat stress, high salinity stress, shade stress and stress due to low nitrogen availability. In another aspect of the present invention, the genetically modified plants have improved subsystem, such as improved plant development, plant morphology, plant physiology or seed composition compared with a corresponding subsystem of a control plant. The various aspects of this invention are particularly useful for genetically modified seeds and genetically modified plants with improved subsystem in maize, soybean, cotton, canola, rapeseed, wheat, sunflower, sorghum, alfalfa, barley, millet, rice, tobacco, fruits and vegetables, and turf. Characterization of the yield traits thus generated will carried out in the fields during at least one season.

### EXAMPLES

The examples below are given so as to illustrate the practice of various embodiments of the present disclosure. They are not intended to limit or define the entire scope of this disclosure. It should be appreciated that the disclosure is not limited to the particular embodiments described and illustrated herein but includes all modifications and variations falling within the scope of the disclosure as defined in the appended embodiments.

### Example 1—Improvement of RuBisCO from Arabidopsis thaliana

In order to form an active Arabidopsis thaliana RuBisCO in yeast, coexpression of 9 genes is performed: RbcL and RbcS subunits and 7 chloroplast chaperones: Cpn60a, Cpn60b, Cpn20, RbcX, Raf1, Raf2 and BSD2 (Aigner et al., 2017). For each gene, different homologous are found after bioinformatic study. Table 1 below identifies paralogs of Rubisco folding and assembly chaperons:

TABLE 1
Paralogs of Rubisco folding and assembly chaperones

| Subunit | MW (kDa) | Arabidopsis |
|---|---|---|
| RbcL | 50-55 | AtCg00490 |
| RbcS | 12-18 | At5g38410 |
| | | At1g67090 |
| | | At5g38420 |
| | | At5g38430 |
| Cpn60α | ~60 | At2g28000(α1) |
| | | At5g18820(α2) |
| Cpn60β | ~60 | At1g55490(β1) |
| | | At3g13470(β2) |
| | | At5g56500(β3) |
| | | At1g26230(β4) |
| Cpn10 | ~10 | At2g44650(1) |
| | | At3g60210(2) |
| Cpn20 | 20-23 | At5g20720 |
| RbcX | ~15 | At4g04330(1) |
| | | At5g19855(2) |
| Raf1 | 40-46 | At5g28500(1) |
| | | At3g04550(2) |
| Raf2 | ~18 | At5g51110 |
| Bsd2 | ~8 | At3g47650 |

For each gene, the different homologous genes are identified after bioinformatics study by comparison with different known RuBisCO subunits (RbcL and RbcS) and assembly chaperonin. To identify the RbcS candidates, an alignment is performed by comparing proteins from Arabidopsis with all the RbcS already described and characterized. Once the genes are identified (Erreur ! Source du renvoi introuvable.), expression of all combinations of homologous genes is performed in yeast in order to reconstruct plant sub-system. Heterologous genes are expressed on plasmid. Heterologous genes may also be expressed from chromosome. For example, the genes RbcL, Cpn60α, Cpn60β and Cpn20 are assembled on transcription unit 1 with or without a selection marker. Transcription unit 1 is inserted into the chromosome of a MATa strain. The genes RbcS, RbcX, Raf1, Raf2 and Bsd2 are assembled on transcription unit 2 with or without a selection marker. Transcription unit 2 is inserted into the chromosome of a MATa strain.

A crossing of MATa and MATa strains is performed and a haploid strain EQ01 with both transcription units 1 and 2 is selected. A directed evolution is carried out on different genes of transcription unit 1 and/or transcription unit 2 by MORPHING as described in Vifla-Gonzalez et al., 2016. A screening of mutants is performed in order to determine the best combination of mutations for RuBisCO activity. The screening is performed by colorimetric assay. The screening is performed by fluorescence following and FACS. The best combination of mutations previously determined is integrated into a yeast engineered in order to constrain the yeast metabolism to use the protein expressed by the combined genes. Adaptive Laboratory Evolution is carried out in chemostat cultivation. To improve mutation generation Ethyl Methane Sulfate is added. Once the best combination of gene mutants is identified, each mutation is introduced into the plant. Evaluation of yield of the plant comprising the mutations versus a plant without the mutations is carried out. RuBisCO activity, photosynthesis yield, growth rate, biomass and seed yield are evaluated. Field evaluation for the plant with mutation is carried out during at least one season.

### Example 2—Improvement of RuBisCO from Arabidopsis thaliana by Directed Mutagenesis of RbcL Subunit

In strain EQ01 obtained in Example 1, gene RbcL is deleted on chromosome and a strain EQ02 is selected. In strain EQ02, RbcL mutants or RbcL wild-type are expressed on plasmid. A screening of mutants is performed in order to determine the best mutations for RuBisCO activity in comparison with the activity obtained with RbcL wild-type as control. Once the best combination of gene mutants is identified, each mutation is introduced into the plant. Evaluation of yield of the plant comprising the mutations versus a plant without the mutations is carried out. RuBisCO activity, photosynthesis yield, growth rate, biomass and seed yield are evaluated. Field evaluation for the plant with mutation is carried out during at least one season.

### Example 3—Improvement of RuBisCO from Arabidopsis thaliana by Directed Mutagenesis of RbcS Subunit

In strain EQ01 obtained in Example 1, gene RbcS is deleted on chromosome and a strain EQ03 is selected. In strain EQ03, RbcS mutants or RbcS wild-type are expressed on plasmid. A screening of mutants is performed in order to determine the best mutations for RuBisCO activity in comparison with the activity obtained with RbcS wild-type as control. Once the best combination of gene mutants is identified, each mutation is introduced into the plant. Evaluation of yield of the plant comprising the mutations versus a plant without the mutations is carried out. RuBisCO activity, photosynthesis yield, growth rate, biomass and seed yield are evaluated. Field evaluation for the plant with mutation is carried out during at least one season.

### Example 4—Improvement of RuBisCO from Arabidopsis thaliana by Directed Mutagenesis of RbcL and RbcS Subunit

In strain EQ02 obtained in Example 2, gene RbcS is deleted on chromosome and a strain EQ04 is selected. In strain EQ04, RbcL mutants or RbcL wild-type are expressed on a first plasmid and RbcS mutants or RbcS wild-type are expressed on a second plasmid. A screening of mutants is performed in order to determine the best mutations for RuBisCO activity in comparison with the activity obtained with RbcL and RbcS wild-type as control. Once the best combination of gene mutants is identified, each mutation is introduced into the plant. Evaluation of yield of the plant comprising the mutations versus a plant without the mutations is carried out. RuBisCO activity, photosynthesis yield, growth rate, biomass and seed yield are evaluated. Field evaluation for the plant with mutation is carried out during at least one season.

### Example 5—Improvement of Phosphate Uptake Kinetics of PHT1;1 and PHT1;4 Transporters from Arabidopsis thaliana

PHT1 phosphate transporters in Arabidopsis thaliana are composed of 9 genes. Table 2 below identifies PHT1 transporters from Arabidopsis thaliana:

TABLE 2
PHT1 transporters from Arabidopsis thaliana

| Name | Length (aa) | Arabidopsis |
|---|---|---|
| PHT1;1 | 524 | At5g43350 |
| PHT1;2 | 524 | AT5G43370 |
| PHT1;3 | 521 | At5g43360 |
| PHT1;4 | 534 | At2g38940 |
| PHT1;5 | 535 | At2g32830 |
| PHT1;6 | 542 | At5g43340 |
| PHT1;7 | 516 | At3g54700 |
| PHT1;8 | 534 | At1g20860 |
| PHT1;9 | 532 | At1g76430 |

In order to form an active Arabidopsis thaliana phosphate transporter in yeast, co-expression of 2 genes is carried out: PHT1;1 and PHT1;4. In order to screen for phosphate uptake efficiency, deletion of PHO84, an endogenous high-affinity phosphate transporter is carried out in S. cerevisiae and a strain EQ05 is selected. The genetic constructions are carried out in the PHO84 mutant yeast strain (EQ5). Because PHO84 has been deleted, EQ05 strain presents a weak growth in low-phosphate medium. Heterologous genes PHT1;1 and PHT1;4 are expressed in EQ05 and a strain EQ06 is selected. EQ06 strain is able to grow much faster in low-phosphate medium than EQ05 strain. Heterologous genes PHT1;1 and/or PHT1;4 are expressed on plasmid or on chromosome. Mutants of heterologous genes PHT1;1 and/or PHT1;4 are synthesized or produced by error prone PCR. These mutants are expressed in EQ05. The strains expressing mutants of PHT1;1 and/or PHT1;4 are cultivated on low-phosphate medium. Strains exhibiting better growth rate than EQ06 are selected. The mutants are sequenced and each mutation is introduced into the plant. Evaluation of yield of the plant comprising the mutations versus a plant without the mutations is carried out. Growth rate, biomass and seed yield are evaluated. Field evaluation for the plant with mutation is carried out during at least one season.

### Example 6—Improvement of Phosphate Uptake Kinetics of PHT1;1, PHT1;2, PHT1;3 and PHT1;4 Transporters from Arabidopsis thaliana

Heterologous genes PHT1;1, PHT1;2, PHT1;3 and PHT1;4 (see Table 2) are expressed in EQ05 and a strain EQ07 is selected. EQ07 strain is able to grow much faster in low-phosphate medium than EQ05 strain. Heterologous genes PHT1;1, PHT1;2, PHT1;3 and PHT1;4 are expressed on plasmid or on chromosome. Mutants of heterologous genes PHT1;1, PHT1;2, PHT1;3 and PHT1;4 are synthesized or produced by error prone PCR. These mutants are expressed in EQ05. The strains expressing mutants are cultivated on low-phosphate medium. Strains exhibiting better growth rate than EQ07 are selected. The mutants are sequenced and each mutation is introduced into the plant. Evaluation of yield of the plant comprising the mutations versus a plant without the mutations is carried out. Growth rate, biomass and seed yield are evaluated. Field evaluation for the plant with mutation is carried out during at least one season.

### Example 7—Improvement of Nitrogenase Activity from Azotobacter vinelandii

In order to form an active Azotobacter vinelandii nitrogenase in yeast, co-expression of 9 genes is performed: nifH, nifD, nifK, nifU, nifS, nifM, nifB, nifE, and nifN. Table 3 below identifies nitrogenase genes from Azotobacter vinelandii

TABLE 3
Genes of component of nitrogenase complex
from Azotobacter vinelandii

| Name | Azotobacter vinelandii |
|---|---|
| nifB | AAA22148.1 |
| nifD | AAA64710.1 |
| nifE | AAA64716.1 |
| nifF | AAA64735.1 |
| nifH | AAA64709.1 |
| nifK | AAA64711.1 |
| nifM | AAA64732.1 |
| nifN | AAA64717.1 |
| nifQ | AAA22151.1 |
| nifS | AAA22168.1 |
| nifU | AAA64725.1 |
| nifV | AAA22169.1 |
| nifX | AAA64718.1 |
| nifY | AAA64713.1 |
| nifZ | AAA64731.1 |

nifH, nifD, nifK, nifU, nifS, nifM, nifB, nifE, and nifN heterologous genes are expressed on plasmid. nifH, nifD, nifK, nifU, nifS, nifM, nifB, nifE, and nifN heterologous genes may also be expressed into a chromosome. For example, the genes nifH, nifD, nifK and nifU are assembled on transcription unit 1 with or without a selection marker. Transcription unit 1 is inserted into the chromosome of a MATa strain. The genes nifS, nifM, nifB, nifE, and nifN are assembled on transcription unit 2 with or without a selection marker. Transcription unit 2 is inserted into the chromosome of a MATa strain. A crossing of MATa and MATa strains is performed and a haploid strain EQ08 with both transcription units 1 and 2 is selected. A directed evolution is carried out on different genes of transcription unit 1 and/or transcription unit 2 by MORPHING as described in Viña-Gonzalez et al., 2016. A screening of mutants is performed in order to determine the best combination of mutations for nitrogenase activity. The screening is performed by colorimetric assay to determine the best combination of gene mutants. The improved nitrogenase system integrating the identified mutation(s) is introduced into the plant. Evaluation of yield of the plant comprising the mutations versus a plant without the mutations is carried out. Nitrogenase activity, growth rate, biomass and seed yield are evaluated. Field evaluation for the plant with mutation is carried out during at least one season.

### Example 8—Improvement of Nitrogenase from Azotobacter vinelandii by Directed Mutagenesis of nifD

In strain EQ08 obtained in Example 7, gene nifD is deleted on chromosome and a strain EQ09 is selected. In strain EQ09, nifD mutants or nifD wild-type are expressed on plasmid. A screening of mutants is performed in order to determine the best mutations for nitrogenase activity in comparison with the activity obtained with nifD wild-type as control. The improved nitrogenase system integrating the identified mutation(s) is introduced into the plant. Evaluation of yield of the plant comprising the mutations versus a plant without the mutations is carried out. Nitrogenase activity, growth rate, biomass and seed yield are evaluated. Field evaluation for the plant with mutation is carried out during at least one season.

### Example 9—Improvement of Nitrogenase from Azotobacter vinelandii by Directed Mutagenesis of nifK

In strain EQ08 obtained in Example 7, gene nifK is deleted on chromosome and a strain EQ10 is selected. In strain EQ10, nifK mutants or nifK wild-type are expressed on plasmid. A screening of mutants is performed in order to determine the best mutations for nitrogenase activity in comparison with the activity obtained with nifK wild-type as control. The improved nitrogenase system integrating the identified mutation(s) is introduced into the plant. Evaluation of yield of the plant comprising the mutations versus a plant without the mutations is carried out. Nitrogenase activity, growth rate, biomass and seed yield are evaluated. Field evaluation for the plant with mutation is carried out during at least one season.

### INCORPORATION BY REFERENCE

All references cited in this specification, and their references, are incorporated by reference herein in their entirety where appropriate for teachings of additional or alternative details, features, and/or technical background.

### EQUIVALENTS

While the disclosure has been particularly shown and described with reference to particular embodiments, it will be appreciated that variations of the above-disclosed and other features and functions, or alternatives thereof, may be desirably combined into many other different systems or applications. Also, that various presently unforeseen or unanticipated alternatives, modifications, variations or improvements therein may be subsequently made by those skilled in the art which are also intended to be encompassed by the following embodiments.

### BIBLIOGRAPHY

Aigner, H., Wilson, R. H., Bracher, A., Claisse, L., Bhat, J. Y., Hartl, F. U., & Hayer-Hartl, M. (2017). Science, 1-8.
Albert, H., Dale, E. C., Lee, E., & Ow, D. W. (1995). The Plant Journal: For Cell and Molecular Biology, 7(4), 649-659.
Altschul, S. F., Madden, T. L., Schäffer, A. A., Zhang, J., Zhang, Z., Miller, W., & Lipman, D. J. (1997). Nucleic Acids Research, 25(17), 3389-3402.
Bieleski, R. L. (1973). Annual Review of Plant Physiology, 24(1), 225-252.
Cai, C. Q., Doyon, Y., Ainley, W. M., Miller, J. C., DeKelver, R. C., Moehle, E. A., Petolino, J. F. (2009). Plant Molecular Biology, 69(6), 699-709.
Corpet, F. (1988). Nucleic Acids Research, 16(22), 10881-10890.
D'Halluin, K., Dittgen, J., Jansens, S., Meulewaeter, F., Weterings, K., Moser, H., Henniger, G. (2013).
D'Halluin, K., Vanderstraeten, C., Van Hulle, J., Rosolowska, J., Van Den Brande, I., Pennewaert, A., Broadhvest, J. (2013). Plant Biotechnology Journal, 11(8), 933-941.
DiCarlo, J. E., Conley, A. J., Penttilä, M., Jäntti, J., Wang, H. H., & Church, G. M. (2013). ACS Synthetic Biology, 2(12), 741-749.
Flamholz, A. I., Prywes, N., Moran, U., Davidi, D., Bar-On, Y. M., Oltrogge, L. M., Milo, R. (2019). Biochemistry, 58(31), 3365-3376.
Feng, Z., Zhang, B., Ding, W., Liu, X., Yang, D. L., Wei, P., . . . Zhu, J. K. (2013). Cell Research, 23(10), 1229-1232.
Gao, F., Shen, X. Z., Jiang, F., Wu, Y., & Han, C. (2016). Nature Biotechnology, 34(7), 768-773.
Gonzalez-Perez, D., Garcia-Ruiz, E., & Alcalde, M. (2012). Bioengineered, 3(3), 174-179.
Grice, J. A., Hughey, R., & Speck, D. (1997). Computer Applications in the Biosciences CABIOS, 13(1), 45-53.
Heidenreich, E. (2007). Critical Reviews in Biochemistry and Molecular Biology, 42(4), 285-311.
Higgins, D. G., & Sharp, P. M. (1988). Gene, 73(1), 237-244.
Higgins, D. G., & Sharp, P. M. (1989). Computer Applications in the Biosciences CABIOS, 5(2), 151-153.
Huang, X., Miller, W., Schwartz, S., & Hardison, R. C. (1992). Bioinformatics, 8(2), 155-165.
Jaganathan, D., Ramasamy, K., Sellamuthu, G., Jayabalan, S., & Venkataraman, G. (2018). Frontiers in Plant Science, 9 (July), 1-17.
Javanpour, A. A., & Liu, C. C. (2019). ACS Synthetic Biology, 8(6), 1249-1256
Jiang, W., Zhou, H., Bi, H., Fromm, M., Yang, B., & Weeks, D. P. (2013). Nucleic Acids Research, 41(20), e188-e188.
Karlin, S., & Altschul, S. F. (1990). Proceedings of the National Academy of Sciences of the United States of America, 87(6), 2264-2268.
Karlin, S., & Altschul, S. F. (1993). Proceedings of the National Academy of Sciences of the United States of America, 90(12), 5873-5877.
Li, X., Wang, Y., Liu, Y., Yang, B., Wang, X., Wei, J., . . . Chen, J. (2018). Nature Biotechnology, 36(4), 324-327.
Li, Z., Xing, A., Moon, B. P., McCardell, R. P., Mills, K., & Falco, S. C. (2009). Plant Physiology, 151(3), 1087-1095.
Lieberman-Lazarovich, M., & Levy, A. A. (2011). In Plant Chromosome Engineering (pp. 51-65).
Lyznik, L A., Gordon-Kamm, W. J., & Tao, Y. (2003). Plant Cell Reports, 21(10), 925-932.
Muchhal, U. S., Pardo, J. M., & Raghotama, K. (1996). 93 (September), 10519-10523. Myers, E. W., & Miller, W. (1988). Bioinformatics, 4(1), 11-17.
Needleman, S. B., & Wunsch, C. D. (1970). Journal of Molecular Biology, 48(3), 443-453.
Nishida, K., Arazoe, T., Yachie, N., Banno, S., Kakimoto, M., Tabata, M., Kondo, A. (2016). Science, 353(6305), aaf8729-aaf8729.
Pearson, W R, & Lipman, D. J. (1988). Proceedings of the National Academy of Sciences of the United States of America, 85(8), 2444-2448.
Pearson, William R. (1994). In Computer Analysis of Sequence Data, Part I (Vol. 24, pp. 307-332).
Podevin, N., Davies, H. V., Hartung, F., Nogué, F., & Casacuberta, J. M. (2013). Trends in Biotechnology, 31(6), 375-383.
Puchta, H. (2002). Plant Molecular Biology, 48, 173-182.
Qi, W., Zhu, T., Tian, Z., Li, C., Zhang, W., & Song, R. (2016). BMC Biotechnology, 16(1), 1-8. Raghothama, K. G. (2005). Plant and Soil, 274(1-2), 37-49.
Ray, D. K., Mueller, N. D., West, P. C., & Foley, J. A. (2013). PLoS ONE, 8(6), e66428.
Renault, S., Duchateau, P., 2012. Site-directed insertion of transgenes. Springer.
Tilman, D., Balzer, C., Hill, J., & Befort, B. L. (2011). Proceedings of the National Academy of Sciences, 108(50), 20260-20264.
Viña-Gonzalez, J., Gonzalez-Perez, D., & Alcalde, M. (2016). Journal of Visualized Experiments, (110), 1-7.
Wheeler, R., & Hughey, R. (2000). Bioinformatics (Oxford, England), 16(12), 1082-1090.
Whitney, S. M., Houtz, R. L., & Alonso, H. (2011). Plant Physiology, 155(1), 27-35.
Wright, D. A., Townsend, J. A., Winfrey, R. J., Irwin, P. A., Rajagopal, J., Lonosky, P. M., Voytas, D. F. (2005). Plant Journal, 44(4), 693-705.
Yau, Y.-Y., Wang, Y., Thomson, J. G., & Ow, D. W. (2011). In Plant Chromosome Engineering (pp. 147-166).
Zaidi, S. S.-A., Mahfouz, M. M., & Mansoor, S. (2017). Trends in Plant Science, 22(7), 550-553.
Zetsche, B., Gootenberg, J. S., Abudayyeh, O. O., Slaymaker, I. M., Makarova, K. S., Essletzbichler, P., Zhang, F. (2015). Cell, 163(3), 759-771.
Zetsche, B., Heidenreich, M., Mohanraju, P., Fedorova, I., Kneppers, J., DeGennaro, E. M., Zhang, F. (2016). Nature Biotechnology, 35(1), 31-34.
Zhang, F., Puchta, H., Thomson, J. G., 2015. Advances in new technology for targeted modification of plant genomes.

1. A method for generating yield traits to improve crop yield, the method comprising:
a) identifying a multi-genic system associated with one or more gene of interest, wherein the one or more gene of interest is operatively linked to yield of crop;
b) identifying at least one combination of genes within the multi-genic system that yields an active multi-protein system of interest;
c) generating mutational diversity on the at least one combination of genes to generate a library;
d) screening the library to identify mutations yielding an improved activity of multi-protein system of interest in an expression system; and
e) introducing the screened mutations of step d) into the crop.

2. The method of claim 1, further comprising expressing in the expression system, a plurality of combinations of genes from crop that are homologous to the multi-genic system to obtain an initial library.

3. The method of claim 1, further comprising screening the first library for the identification of the at least one combination of genes within the multi-genic system.

4. The method of claim 1, wherein the generation of mutational diversity is performed by site-directed mutagenesis.

5. The method of claim 2, wherein the expression system is yeast.

6. The method of claim 5, wherein the multi-genic system is inserted into one or different chromosomes in the yeast.

7. The method of claim 5, wherein the multi-genic system is expressed on one or more plasmids.

8. The method of claim 1, wherein the crop yield traits is carbon fixation.

9. The method of claim 1, wherein the multi-genic system is a RuBisCo system.

10. The method of claim 9, wherein the RuBisCo system comprises subunit L, subunit S and chaperonins.

11. The method of claim 10, wherein the chaperonins comprise Cpn60α, Cpn60β, Cpn20, RbcX, Raf1 Raf2 and Bsd2.

12. A method for generating yield traits to improve crop yield, the method comprising:
a) identifying a multi-genic system associated with one or more gene of interest, wherein the one or more gene of interest is operatively linked to crop yield;
b) expressing in an expression system a plurality of combinations of genes from crop that are homologous to the multi-genic system to obtain a first library;
c) screening the first library for at least one combination of genes yielding the active multi-genic system of interest in the expression system;
d) integrating the at least one combination of genes in the expression system to carry out an adaptive laboratory evolution and to generate an evolved population of the expression system;
e) screening the evolved population to identify mutations resulting in an improved activity of the multi-genic system of interest; and
f) introducing the screened mutations of step e) into the crop.

13. The method of claim 12, wherein the expression system is yeast.

14. The method of claim 13, wherein the multi-genic system is inserted into one or different chromosomes in the yeast.

15. The method of claim 13, wherein the multi-genic system is expressed on one or more plasmids.

16. The method of claim 12, wherein the crop yield traits is carbon fixation.

17. The method of claim 12, wherein the multi-genic system is a RuBisCo system.

18. The method of claim 17, wherein the RuBisCo system comprises subunit L, subunit S and chaperonins.

19. The method of claim 18, wherein the chaperonins comprise Cpn60α, Cpn60β, Cpn20, RbcX, Raf1 Raf2 and Bsd2.
